(19) United States
(12) Patent Application Publication
(10) Pub. No.: US 2007/0244371 A1
(43) Pub. Date: Oct. 18, 2007

(54) Phlebectomy illumination device and methods

(76) Inventors: Hoa D. Nguyen, San Jose, CA (US); Michael S. Mirizzi, San Jose, CA (US); Brian E. Farley, Los Altos, CA (US); Arthur W. Zikorus, San Jose, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/732,771

(22) Filed: Apr. 4, 2007

Related U.S. Application Data
(60) Provisional application No. 60/744,266, filed on Apr. 4, 2006.

(51) Int. Cl. A61B 5/00

(52) U.S. Cl. 600/300

(57) ABSTRACT

The disclosure relates to the use of subcutaneous illumination during phlebectomy procedures, and to devices and methods for performing hook phlebectomy while visualizing veins and surrounding tissue using light emitted subcutaneously and beneath the surgical area. An apparatus comprises, in one embodiment, a handle, an illuminator portion extending distally from the handle, the illuminator portion comprising an elongate member configured for insertion into subcutaneous tissue, the illuminator portion comprising an elongate electroluminescent device extending along the illuminator portion, the electroluminescent device being configured to generate and emit light primarily in a generally radial direction, outward and away from a longitudinal axis of the illuminator portion. Furthermore, a phlebectomy hook is provided that comprises, in one embodiment, a shaft, a bend extending generally laterally from the shaft, the bend terminating in a tip of the phlebectomy hook, and an illuminator, the illuminator configured to propagate a visible light beam near the bend, generally outward relative to a longitudinal axis of the shaft.

### CROSS-REFERENCE TO RELATED APPLICATIONS

This application claims priority to U.S. Provisional Patent Application No. 60/744,266, filed Apr. 4, 2006, titled PHLEBECTOMY ILLUMINATION, which is hereby incorporated by reference in its entirety and made part of this specification.

### BACKGROUND

1. Field

The present disclosure relates to the use of subcutaneous illumination during phlebectomy procedures, and to devices and methods for performing hook phlebectomy while visualizing veins and surrounding tissue using light emitted subcutaneously and beneath the surgical area.

2. Description of the Related Art

Phlebectomy is a standard treatment for varicosities arising from incompetent veins, particularly those below the saphenofemoral and saphenopopliteal junctions. This technique involves the avulsion and removal of the varicose veins through multiple stab incisions made directly over the veins, which are removed using surgical tools such as hooks and mosquito forceps.

It is essential in performing phlebectomy that the surgeon be able to properly locate the veins to be removed, so that the incisions are properly situated. In general, this is accomplished by proper preoperative location and marking of the veins to be removed. This is generally accomplished with the patient in a standing position so that the veins become engorged with blood due to the action of gravity and thus easily marked. The vein locations are marked with an indelible pen or other surgical marker. However, the operation is generally conducted with the patient in a supine position. The position of the incompetent veins may shift during the transition from a standing to a supine position, and often further marking is conducted after the patient is placed in the surgical position in an attempt to track these shifts.

Once the patient has been marked, the patient has been placed in the supine operative position, and incisions have been made along the length of the vein to be removed, the surgeon then probes with the surgical implements, such as hooks, to locate and remove the vein. In some cases, it is difficult to quickly distinguish the vein from the surrounding tissue when working through small surgical incisions such as those commonly employed in phlebectomy. This is especially the case when, as often occurs, the position of the veins to be removed has shifted from the marked positions.

### SUMMARY

In one embodiment, an apparatus is provided for subcutaneous illumination of a blood vessel, the apparatus comprising: a handle; an illuminator portion extending distally from the handle, the illuminator portion comprising an elongate member configured for insertion into subcutaneous tissue; the illuminator portion comprising an elongate electroluminescent device extending along the illuminator portion, the electroluminescent device being configured to generate and emit light primarily in a generally radial direction, outward and away from a longitudinal axis of the illuminator portion.

In a further optional aspect of the apparatus, the electroluminescent device comprises at least one LED.

In another optional aspect of the apparatus, the electroluminescent device comprises a linear array of solid state light emitters. Such solid state light emitters can optionally comprise LEDs.

In a further optional aspect of the apparatus, the illuminator portion comprises a flattened bladelike member of about 2.5-7.5 centimeters in length.

In a further optional aspect of the apparatus, the illuminator portion comprises a flattened bladelike member less than about 3 mm thick.

In a further optional aspect of the apparatus, the illuminator portion comprises a flattened bladelike member configured to penetrate and dissect subcutaneous tissue.

In a further optional aspect of the apparatus, the illuminator portion is configured to provide visible light substantially only in a wavelength or wavelength range which tends to highlight a blood vessel against surrounding tissue when passed therethrough.

In a further optional aspect of the apparatus, the illuminator portion is configured to provide substantially only yellow or amber light.

In a further optional aspect of the apparatus, the illuminator portion is bent relative to a longitudinal axis of the handle.

In a further optional aspect of the apparatus, a bendable connector member is provided which connects the illuminator portion to the handle, the connector member being configured to retain a bent shape imparted to it.

In another embodiment, an apparatus for subcutaneous illumination of a blood vessel is provided, the apparatus comprising: a handle; an illuminator portion extending distally from the handle, the illuminator portion comprising an elongate member configured for insertion into subcutaneous tissue; the illuminator portion being configured to propagate light primarily in a generally radial direction, outward and away from a longitudinal axis of the illuminator portion; the illuminator portion being further configured to propagate visible light substantially only in a wavelength or wavelength range selected to highlight a blood vessel against surrounding tissue when passed therethrough.

In a further optional aspect of the apparatus, the illuminator portion is configured to propagate visible light substantially only in the yellow or amber range.

In a further optional aspect of the apparatus, the illuminator portion is configured to propagate visible light substantially only around a wavelength below 610 nm.

In a further optional aspect of the apparatus, the illuminator portion comprises an electroluminescent device configured to emit visible light substantially only in the selected wavelength or wavelength range. The electroluminescent device can optionally comprise a linear array of solid state light emitters. Such solid state light emitters can optionally comprise LEDs.

In a further optional aspect of the apparatus, the apparatus also comprises a light source configured to emit light; wherein the illuminator portion comprises a light conductor configured to conduct light from the light source and direct the light in the radial direction.

In a further embodiment, a method of enhancing visibility of a blood vessel in subcutaneous tissue is provided, the method comprising: passing light through the tissue and the blood vessel; with the light, highlighting the blood vessel against a surrounding portion of the subcutaneous tissue.

In a further optional aspect of the method, the highlighting comprises causing the blood vessel to appear substantially black. The highlighting can further optionally comprise causing the surrounding portion of the tissue to appear lighter than black, which can optionally comprise causing the surrounding portion of the tissue to appear substantially yellow. The blood vessel can optionally comprise a vein.

In a further optional aspect of the method, passing light comprises passing substantially only yellow or amber light.

In a further optional aspect of the method, passing light comprises passing substantially only light with a wavelength below 610 nm.

In a further optional aspect of the method, the method further comprises inserting a light instrument into the subcutaneous tissue; wherein the passing light comprises passing light from the light instrument, through the subcutaneous tissue and toward an observer.

In a further optional aspect of the method, highlighting the blood vessel comprises increasing visible contrast between the blood vessel and the surrounding portion of the subcutaneous tissue. Such increasing the visible contrast can optionally comprise passing the light substantially only at a wavelength or wavelength band which tends to darken the blood vessel to a greater degree than the surrounding portion of the subcutaneous tissue. The blood vessel can optionally comprise a vein and the wavelength band can comprise visible light below 610 nm, or yellow or amber light.

In a further embodiment, a phlebectomy hook is provided that comprises: a shaft; a bend extending generally laterally from the shaft, the bend terminating in a tip of the phlebectomy hook; and an illuminator, the illuminator configured to propagate a visible light beam near the bend, generally outward relative to a longitudinal axis of the shaft.

In a further optional aspect of the phlebectomy hook, the shaft has a proximal end and a distal end; the bend is located at the distal end; and the illuminator is configured to propagate the visible light proximally and outward relative to the longitudinal axis. The illuminator can further optionally be configured to propagate the visible light beam along a light path extending from a junction of the bend and the shaft outward past the tip.

In a further optional aspect of the phlebectomy hook, the illuminator is configured to propagate the visible light beam along a light path extending from a junction of the bend and the shaft outward past the tip.

In a further optional aspect of the phlebectomy hook, the illuminator is configured to propagate visible light substantially only in the yellow or amber range.

In a further optional aspect of the phlebectomy hook, the illuminator comprises a light conductor having a reflective surface near the bend, the light conductor being configured to propagate the visible light beam from the reflective surface.

In a further optional aspect of the phlebectomy hook, the illuminator comprises a light source.

In a further optional aspect of the phlebectomy hook, the illuminator comprises an electroluminescent device. The electroluminescent device can optionally comprise an LED.

In a further embodiment, a phlebectomy method is provided that comprises: inserting a phlebectomy hook into tissue near a vein; and enhancing visibility of the vein by shining light from an illuminator of the phlebectomy hook through the tissue and the vein, toward and past the skin surface.

In a further optional aspect of the method, the method further comprises pulling a portion of the vein toward the skin surface with the phlebectomy hook.

In a further optional aspect of the method, enhancing visibility of the vein comprises increasing visible contrast between the vein and the tissue. Such increasing the visible contrast can optionally comprise shining the light substantially only at a wavelength or wavelength band which tends to darken the vein to a greater degree than the tissue. The wavelength band can optionally comprise visible light below 610 nm, or yellow or amber light.

In a further optional aspect of the method, shining light comprises shining visible light substantially only at a wavelength or wavelength band which tends to darken the vein to a greater degree than the tissue.

In a further optional aspect of the method, shining light comprises shining visible light substantially only in the yellow or amber range.

In a further optional aspect of the method, shining light comprises shining visible light substantially only below 610 nm.

### BRIEF DESCRIPTION OF THE DRAWINGS

FIG. 1 illustrates an apparatus for subcutaneous illumination of a blood vessel;

FIG. 2 shows the apparatus of FIG. 1 with the illuminator portion bent with respect to the handle;

FIGS. 3A and 3B show, respectively, a plan view and a cross-sectional view of an exemplary electroluminescent device;

FIG. 4 illustrates another embodiment of an apparatus for subcutaneous illumination of a blood vessel;

FIGS. 5A and 5B show side and cross-sectional views of the distal end of the device of FIG. 4; and

FIG. 6 shows a side view of the distal end of a phlebectomy hook with an illuminator for subcutaneously illuminating a blood vessel.

### DETAILED DESCRIPTION OF THE PREFERRED EMBODIMENTS

The present disclosure relates to the visualization of veins and surrounding tissue using light supplied subcutaneously from, for example, a device used to avulse or remove incompetent veins during phlebectomy procedures. The devices can incorporate an illumination system that can be deployed subcutaneously deep below the veins, e.g., through small phlebectomy incisions. In general, the veins will appear as dark shadows behind the skin or as darker than the surrounding tissue.

Visualization of Tissue via Subcutaneous Illumination

Improved differentiation can be achieved by selecting illuminating wavelength(s) (i.e. color(s)) that can provide enhancement of specific anatomical structures. Principles of color addition and subtraction will determine the colors that will be absorbed, and those that will be reflected and scattered. The colors absorbed differ depending on the chemical makeup of the tissues illuminated. For example, the absorption of green light by hemoglobin in blood is about twenty times as great as the absorption of red light. A much higher proportion of incident red light will thus be reflected and scattered by blood. Selecting an illumination source which will reduce the amount of reflected red would therefore be advantageous. A suitable filter applied to the broadband light of a quartz halogen illuminator, or an inherently narrowband light source such as a LED or laser, could be employed.

Yellow light is composed of red and green light. Illumination of vasculature with yellow light results in preferential absorption of the green light and reflection of the red light. The red light reflection will decrease as the blood becomes deoxygenated. Nevertheless, because of the propensity of blood to reflect some incident red light, it has been found advisable to reduce the amount of red light emitted by the illumination source.

Light having wavelengths shorter than approximately 610 nm appears yellow or amber in color and excludes a significant portion of the visible red light spectrum. When such yellow light is used to illuminate veins and surrounding tissue, the reflected and scattered red light can be greatly reduced. Such illumination results in the absorption of the green component and most of the remaining red component of the illuminating light, with very little light reflection. Under such illumination, veins appear black, with surrounding tissue appearing lighter than black or yellow. Consequently, for red (oxygenated vasculature), green is absorbed and red is reflected. For blue (deoxygenated vasculature), green and red are absorbed and nothing is reflected, black is observed.

The wavelengths that create the best contrast between veins and surrounding tissue when used in phlebectomy procedures were determined as follows. A graph of absorption coefficient versus wavelength indicates that at near infrared wavelengths in the 750-900 nm region, absorption of light by oxygenated and deoxygenated hemoglobin is orders of magnitude greater than the absorption coefficient of water. As tissue is largely composed of water, this difference would indicate that infrared light would pass through tissue while being absorbed by blood.

The difference in absorption allows light to pass through derma, muscle, and fat unobstructed except by hemoglobin. It was experimentally determined that a simple LED illuminator with a peak spectral wavelength of 880 nm is adequate for transilluminating the veins in a human finger.

Infrared is not in the visible spectrum and therefore cannot be seen without an imaging modality such as a CCD camera. A Sony Night Shot camera with an infrared viewing mode was used in testing. Greater intensity was obtained for transdermal illumination by using high power infrared LED arrays with a peak spectral wavelength of 880 nm. Difficulty in collecting light from these arrays for introduction into a fiber optic resulted in using a more standard light source modified for the purpose. Adequate illumination of the subcutaneous tissues was obtained by filtering a quartz halogen fiberoptic light source with a long wave filter (cutoff at 750 nm). The addition of a long pass filter to the Sony Night Shot camera, eliminating much of the visible background light, allowed operation under normal room lighting. Filtering out of band (other than infrared) light also resulted in an improvement in the signal to noise ratio, producing clearer images with less noise.

The light may be applied transdermally or introduced subcutaneously, either using a light emitting device that is inserted subcutaneously or using a device that conveys light subcutaneously via fiber optics. Both methods have clinical utility. As described below, tumescent fluid or saline fluid is injected subcutaneously to increase the field of illumination. When light is applied transdermally to image veins prior to a phlebectomy procedure, as when for example a patient is placed in a supine position and an assessment is made as to whether the veins to be removed have shifted from the positions marked in the standing position, high power LED arrays may be applied to the skin. Emission of yellow or amber light having a reduced red component will cause the veins to appear as dark shadows against the skin, permitting reassessment and adjustment of the markings prior to the initial incisions.

Embodiments of the Illumination Device

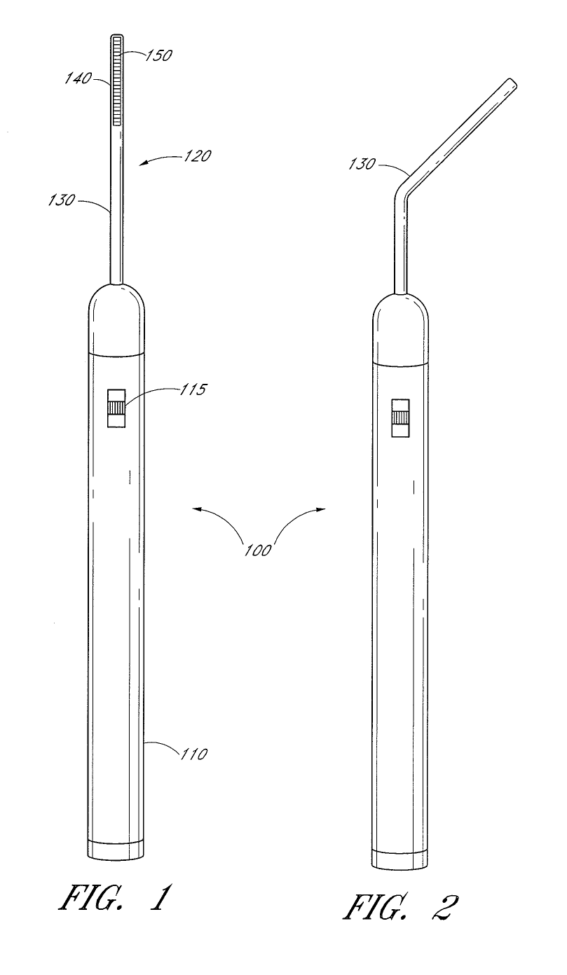

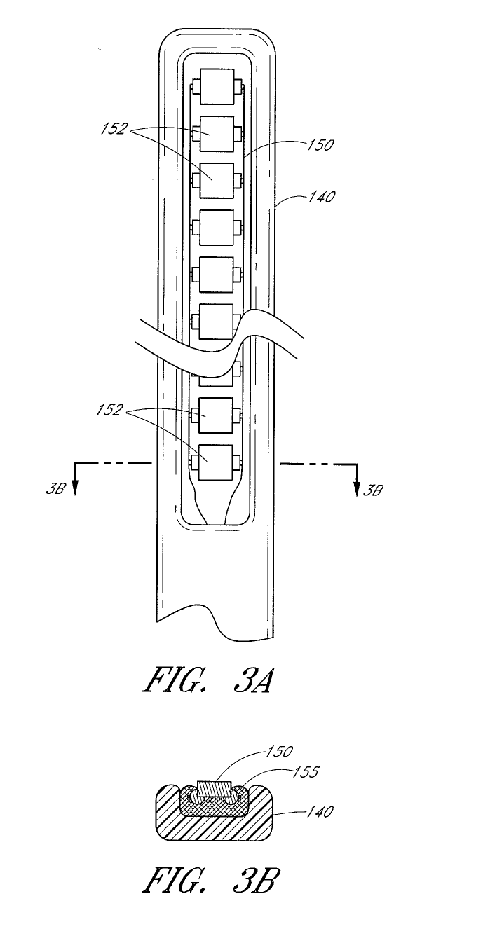

A first embodiment of an apparatus 100 for subcutaneous illumination of blood vessels is shown in FIG. 1. This apparatus comprises a handle 110 and an illuminator portion 120. The handle 110 will generally comprise a switch 115 permitting the surgeon to turn the electroluminescent device 150 off and on. As shown in FIG. 1, the illuminator portion 120 comprises an elongated shaft 130 and a flattened blade or bladelike member 140 at the distal end of shaft 130. In certain embodiments, the shaft 130 may be of sufficient stiffness to permit the dissection of tissue or the avulsing of veins using the blade 140. The shaft 130 may also be bendable at one or more positions, so that the longitudinal axis of the illuminator portion, or some part thereof, diverges from the longitudinal axis of the handle or of the device generally. For example, a bendable connector member may be provided between the illuminator portion and the handle, which is configured to retain a bent shape imparted to it and has sufficient strength to permit the use of the illuminator portion to penetrate and dissect tissue irrespective of the position of the bendable connector member. Alternatively, the illuminator portion 120 may be bendable over the entirety of the length thereof. A subcutaneous illumination apparatus with a bent shaft 130 is depicted in FIG. 2.

The blade 140 is of a size that permits insertion to the subcutaneous surgical space through the small stab incisions typically employed in phlebectomy. Typically, the blade 140 will be within a range of 2.5-7.5 cm in length, more preferably within a range of 3.5-6 cm in length, and most preferably approximately 4 cm in length. The width of the blade is set so as to permit insertion into a small stab incision, while accommodating the electroluminescent devices described below. Preferably, the width is within a range of 2-5 mm, more preferably within a range of 3-4.5 mm, and most preferably approximately 3 mm. The blade should have a sufficient thickness to impart the sturdiness necessary to dissect tissue, while avoiding excessive thickness that would impede the process of dissection or cause excessive damage to surrounding tissues. Preferably, the thickness is less than approximately 3 mm, more preferably within a range of 1-2.5 mm, and most preferably approximately 2 mm.

The shaft 130 and blade 140 should comprise a biocompatible material having sufficient strength to permit the dissection of tissue using the blade 140. In certain embodiments, biocompatible metals may be employed. Stainless steel is preferentially used, although alloys of other metals may also be employed, such as titanium alloys like Nitinol or cobalt alloys. Alternatively, polymer-based materials may also be employed, provided they have sufficient strength.

The blade 140 incorporates an elongate electroluminescent device 150 which is configured to both generate and emit light in a generally radial direction, outward and away from a longitudinal axis of the illuminator portion. Any suitable type of electroluminescent lighting apparatus that may be accommodated within the blade 140 may be employed in the device 150. Examples thereof include LED elements and other solid state light emitters. Individual light emitting elements may be assembled into a linear array as seen in FIG. 1 to form the device 150. In one particular embodiment, the light emitting elements may comprise a linear array of SMD super bright LED elements. Known useful types of LEDs include part no. 475-1157-1-ND, available from Digi-Key Corporation of Thief River Falls, Minn., in a MINITOPLED package and driven with a 20 mA forward current; or Digi-Key part no. 475-1187-1-ND, in a 4-PLCC SMD package and driven with a 30 mA forward current. In certain embodiments, the light emitting elements are configured to emit yellow light. In other embodiments, the light emitting elements are configured to emit light having a wavelength of less than 610 nm. In other embodiments, the light emitting elements are configured to emit light substantially only in a wavelength or wavelength range that tends to highlight a blood vessel against surrounding tissue when the light is passed through the blood vessel and tissue. In other words, the incident light causes the vein to appear darker than the surrounding tissue. The light emitting elements can be powered by batteries or other energy storage devices that are housed within handle 110, or by an external power supply to which the apparatus 100 may be connected such as a wall outlet.

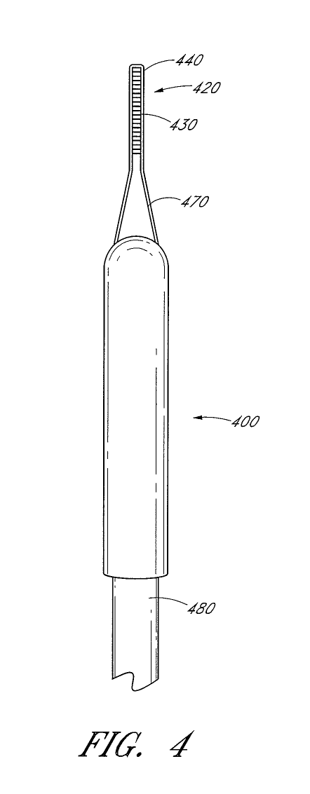

Plan and cross-sectional views of one example of an electroluminescent device 150, comprising a linear arrangement of individual light emitting elements 152, are shown in FIGS. 3A-3B. The cross-sectional view shown in FIG. 3B is taken along the line A-A of FIG. 3A. As shown in FIG. 3B, a filling material 155 fills the gap between the electroluminescent device 150 and the blade 140. The material 155 may be a flowable thermosetting polymer such as an epoxy resin.

The electroluminescent device 150 may also comprise a biocompatible coating placed over the device 150 or over the blade 140 as well. The coating can serve to facilitate the passage of the device 150 or blade 140 through the tissue to be dissected, as well as to protect and insulate the light emitting element or elements within the device 150. The coating may be selected for its light transmission properties as well as its protective properties. In certain embodiments, clear biocompatible acrylic polymer is employed for the coating. In other embodiments, the material employed in the coating may be selected to function as a light filter and preferentially transmit certain wavelengths of light, such as yellow light or light having a wavelength of 610 nm or less.

This embodiment of the apparatus 100 for subcutaneous illumination may be employed as follows. Commonly, phlebectomy procedures involve the use of tumescent anesthesia, using for example large-volume, low-concentration lidocaine. Subcutaneous application of the tumescing solution elevates the veins closer to the skin surface and increases the field of illumination, as described above. Where the standard anesthesia protocol is inadequate to provide the desired conditions for illumination, additional saline solution may be injected.

Holding the handle 110 of the apparatus 100, the surgeon inserts the illuminator portion 120 through a skin incision made in the vicinity of a vein to be avulsed. The surgeon may engage the electroluminescent device 150 using switch 115 either before inserting the illuminator portion 120 or after insertion. The handle 110 is then manipulated to place the apparatus beneath a vein to be avulsed, with the electroluminescent device 150 facing upward. The light emitted by the electroluminescent device 150 then passes upward through the deoxygenated blood in the vein and the surrounding tissue, enabling the surgeon to better visualize the vein. For example, where the electroluminescent device 150 emits yellow or amber light, or light having a wavelength of approximately 610 nm or less, the vein will appear black in contrast to the surrounding tissue, which will appear yellow. When viewed through the skin, the vein will appear as a dark shadow, facilitating location and the placement of further incisions, if required.

For the initial location of the vein, it is preferred that the longitudinal axis of the electroluminescent device 150 be oriented transverse to the presumed longitudinal axis of the vein. This configuration offers the greatest chance that the vein will fall within the field of illumination of the electroluminescent device 150. Once the vein has been located, the surgeon may employ the blade 140 to locally avulse the vein prior to its removal through the skin incisions. Alternatively, a separate surgical implement may be employed to avulse the vein along its length while it is being visualized through the skin.

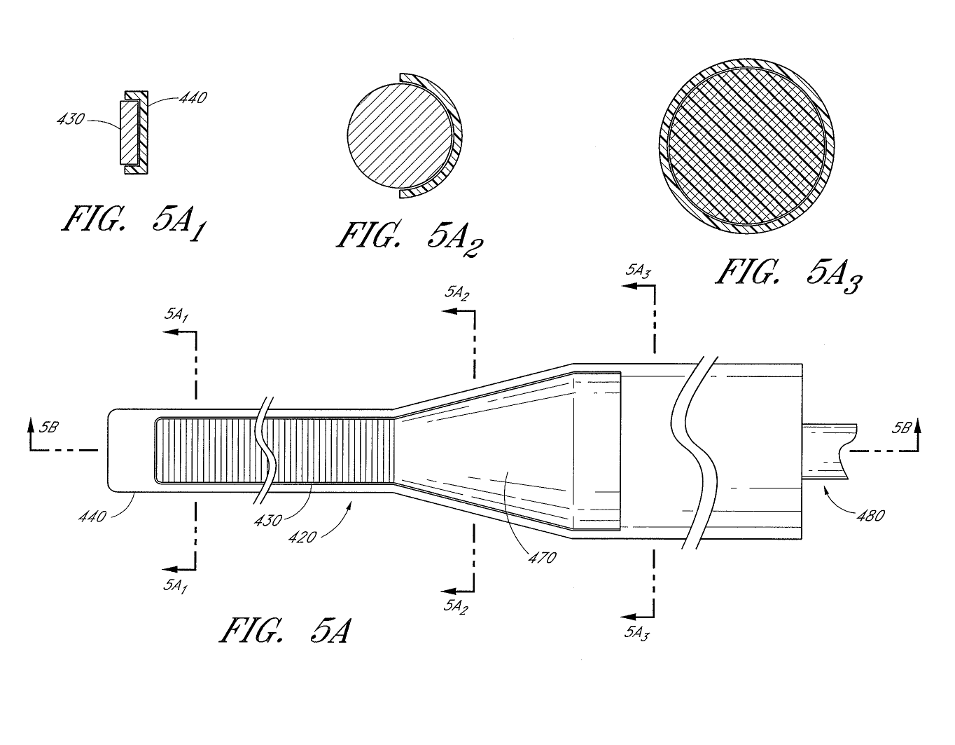

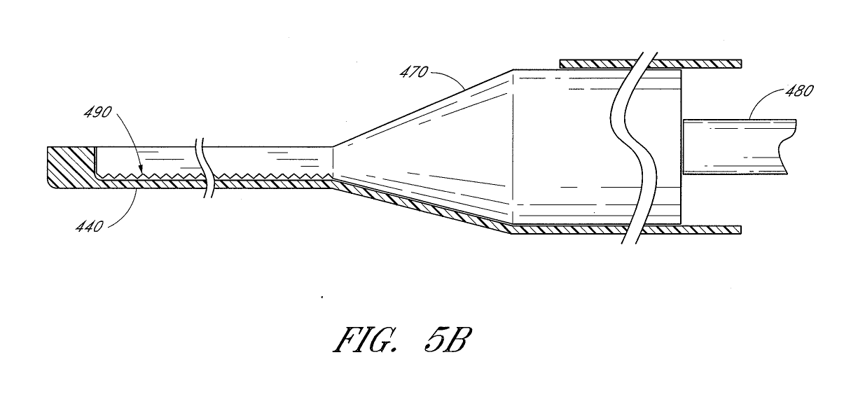

A second embodiment of a phlebectomy illumination device is depicted in FIG. 4. In this embodiment, which can be generally similar to the apparatus 100 except as further described herein, the device 400 incorporates or is connected to an external illumination source. In this embodiment, as shown in greater detail in FIG. 5, the illuminator portion 420 comprises a glass or optical plastic strip 430 that is connected to an external light source via a light guide 470 and a fiber optic bundle 480. As shown by the cross-sectional views taken along the lines A-A, B-B, and C-C in FIG. 5A, the light guide 470 is a cylindrical rod through most of its length, tapering to a connection with the strip 430, which is flattened.

The strip is supported by a support structure 440 that imparts sufficient strength to the illuminator portion 420 to enable it to be used to dissect tissue. The support structure 440 may be formed of a metal such as stainless steel, a titanium alloy, or a cobalt alloy, or may comprise a plastic material, so long as the plastic material imparts sufficient strength to the illuminator portion 420.

The light guide 470 may comprise an optical plastic such as an acrylic polymer. The fiber optic bundle 480 is optically connected to the light guide 470 at the proximal end of the light guide 470. The fiber optic bundle 480 is connected at the opposite end thereof to a light source. In certain embodiments, the light source may be configured to generate yellow or amber light, or light having a wavelength of 610 nm or less, or light that is selected to highlight a blood vessel against surrounding tissue when the light is passed through the blood vessel and the tissue.

The illuminator portion 420 comprises an axial-to-radial reflector 490. At the tip thereof, this reflector 490 may comprise a polished reflective inner surface of the support structure 440. Alternatively, it may comprise a reflective material affixed to the inner surface of the support structure 440 or the bottom surface of the strip 430. The reflector 490 can also comprise reflecting prisms that are etched on the lower side of the strip 430. The depth of the prisms can increase as the reflector extends distally. The prisms serve to reflect light conveyed from the light source and axially via fiber optic bundle 480, light guide 470, and strip 430 in a radial outward direction.

This embodiment of the apparatus 400 for subcutaneous illumination may be employed in a manner substantially identical to the way in which the device of the first embodiment is employed. The device 400 is manipulated so that the apparatus is located below a vein to be avulsed and the side of illuminator portion 420 on which strip 430 is located, and which emits visible light reflected from reflector 490, faces upward. In this position, the light emitted from illuminator portion 420 will pass through the vein and surrounding tissue, illuminating the vein and improving its visibility to the surgeon. In some embodiments, the wavelength is selected so that the light has a yellow or amber color. An appropriate wavelength in such a case is approximately 610 nm or less. As described above, light having such a wavelength creates a black shadow when it passes through blue deoxygenated blood, based on the principles of color addition and subtraction.

In this embodiment, because the illumination portion contains no light generating elements, but rather simply reflects light generated remotely, the illumination portion itself can be very low profile. This may be advantageous where a finer avulsing implement is desired, as for example in the removal of smaller incompetent veins.

A third embodiment of a device that may be employed to illuminate veins and surrounding tissue during phlebectomy procedures operates on similar illumination principles to the avulsing device shown in the second embodiment, but is in the form of a hook commonly employed in phlebectomy procedures, such as a Muller hook, an Oesch hook, or a Ramelet hook. In this embodiment, light is conveyed axially from a remote light source via an optical fiber and a light guide such as an acrylic rod, and is reflected so as to project approximately radially outward from the tip of the hook.

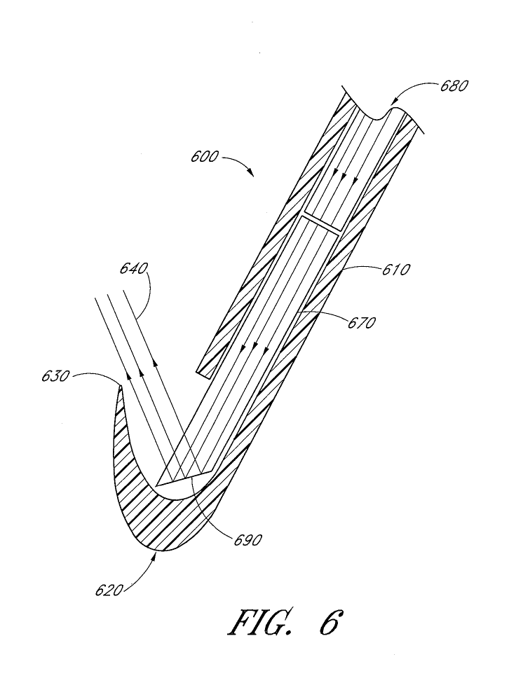

One embodiment of the hook 600 is depicted in FIG. 6. The optical fiber 680 and light guide 670 are contained within the shaft 610 of hook 600, which generally comprises a strong, rigid biocompatible material such as surgical grade steel, although other biocompatible materials may also be employed. The light guide 670 generally comprises an optical plastic rod. The distal end of light guide 670 is cut at an acute angle to the longitudinal axis thereof, forming a beveled end surface 690. As shown in FIG. 6, the angle of the bevel is selected so that light traveling along the light guide 670 will be reflected at the distal beveled end surface 690 and will reflect outward in the same direction as the hook tip 630 that is generally employed to capture the vein. To facilitate reflection of the light by the beveled end surface 690, the surface can be polished or otherwise subjected to a mirroring treatment.

The hook 600 is provided with a bend 620 at the distal or working end thereof; the bend 620 terminates in the tip 630 of the hook 600. The light path 640 of the reflected light extends approximately from the junction of the bend 620 and the shaft 610. The tip 630 may be configured to pierce or otherwise capture a vein to be removed.

In another embodiment, the hook 600 is provided with an illuminator comprising an onboard light emitting element located at the distal end of the hook 600. The light emitting element may comprise one or more LEDs or solid state light emitting elements. The light emitting element may be powered by an integral battery or other power source. The light emitted by the element is propagated outward and serves the same function as the light reflected by the beveled end surface 690 shown in FIG. 6.

This embodiment of a phlebectomy hook employing subcutaneous illumination may be employed in the following manner. The hook 600 is inserted through an incision in the skin and is manipulated by the surgeon so that the end of the hook is located beneath the vein to be removed. The light source is engaged, and the light emitted passes upward through the vein and surrounding tissue to facilitate location of the vein. The light source may have a wavelength of light selected to create a black shadow indicating the vein as viewed through the skin due to the deoxygenated blood by way of the color addition and subtraction principles described earlier. In such a case, the wavelength is a yellow or amber color and is approximately 610 nm in wavelength or less.

Once the vein has been located using the visible light beam emitted from the junction of the hook, the vein is pierced, hooked or otherwise secured using the tip 630 or the internal portion of the bend 620, and is removed through the skin incision together with the hook 600 in the standard manner.

What is claimed is:

1. An apparatus for subcutaneous illumination of a blood vessel, said apparatus comprising:
a handle;
an illuminator portion extending distally from said handle, said illuminator portion comprising an elongate member configured for insertion into subcutaneous tissue;
said illuminator portion comprising an elongate electroluminescent device extending along said illuminator portion, said electroluminescent device being configured to generate and emit light primarily in a generally radial direction, outward and away from a longitudinal axis of said illuminator portion.

2. The apparatus of claim 1, wherein said electroluminescent device comprises at least one LED.

3. The apparatus of claim 1, wherein said electroluminescent device comprises a linear array of solid state light emitters.

4. The apparatus of claim 4, wherein said solid state light emitters comprise LEDs.

5. The apparatus of claim 1, wherein said illuminator portion comprises a flattened bladelike member of about 2.5-7.5 centimeters in length.

6. The apparatus of claim 1, wherein said illuminator portion comprises a flattened bladelike member less than about 3 mm thick.

7. The apparatus of claim 1, wherein said illuminator portion comprises a flattened bladelike member configured to penetrate and dissect subcutaneous tissue.

8. The apparatus of claim 1, wherein said illuminator portion is configured to provide visible light substantially only in a wavelength or wavelength range which tends to highlight a blood vessel against surrounding tissue when passed therethrough.

9. The apparatus of claim 1, wherein said illuminator portion is configured to provide substantially only yellow or amber light.

10. The apparatus of claim 1, wherein said illuminator portion is bent relative to a longitudinal axis of said handle.

11. The apparatus of claim 1, further comprising a bendable connector member which connects said illuminator portion to said handle, said connector member being configured to retain a bent shape imparted to it.

12. A method of enhancing visibility of a blood vessel in subcutaneous tissue, said method comprising:
passing light through said tissue and said blood vessel;
with said light, highlighting said blood vessel against a surrounding portion of said subcutaneous tissue.

13. The method of claim 12, wherein said highlighting comprises causing said blood vessel to appear substantially black.

14. The method of claim 13, wherein said highlighting further comprises causing said surrounding portion of said tissue to appear lighter than black.

15. The method of claim 13, wherein said highlighting further comprises causing said surrounding portion of said tissue to appear substantially yellow.

16. The method of claim 14, wherein said blood vessel is a vein.

17. The method of claim 12, wherein said passing light comprises passing substantially only yellow or amber light.

18. The method of claim 12, wherein said passing light comprises passing substantially only light with a wavelength below 610 nm.

19. The method of claim 12, further comprising inserting a light instrument into said subcutaneous tissue; wherein said passing light comprises passing light from said light instrument, through said subcutaneous tissue and toward an observer.

20. The method of claim 12, wherein highlighting said blood vessel comprises increasing visible contrast between said blood vessel and said surrounding portion of said subcutaneous tissue.

21. The method of claim 20, wherein increasing said visible contrast comprises passing said light substantially only at a wavelength or wavelength band which tends to darken said blood vessel to a greater degree than said surrounding portion of said subcutaneous tissue.

22. The method of claim 21, wherein said blood vessel comprises a vein and said wavelength band comprises visible light below 610 nm.

23. The method of claim 21, wherein said blood vessel comprises a vein and said wavelength band comprises yellow or amber light.

24. A phlebectomy hook comprising:
a shaft;
a bend extending generally laterally from said shaft, said bend terminating in a tip of said phlebectomy hook; and
an illuminator, said illuminator configured to propagate a visible light beam near said bend, generally outward relative to a longitudinal axis of said shaft.

25. The phlebectomy hook of claim 24, wherein:
said shaft has a proximal end and a distal end;
said bend is located at said distal end; and
said illuminator is configured to propagate said visible light proximally and outward relative to said longitudinal axis.

26. The phlebectomy hook of claim 25, wherein said illuminator is configured to propagate said visible light beam along a light path extending from a junction of said bend and said shaft outward past said tip.

27. The phlebectomy hook of claim 24, wherein said illuminator is configured to propagate said visible light beam along a light path extending from a junction of said bend and said shaft outward past said tip.

28. The phlebectomy hook of claim 24, wherein said illuminator is configured to propagate visible light substantially only in the yellow or amber range.

29. The phlebectomy hook of claim 24, wherein said illuminator comprises a light conductor having a reflective surface near said bend, said light conductor being configured to propagate said visible light beam from said reflective surface.

30. The phlebectomy hook of claim 24, wherein said illuminator comprises a light source.

31. The phlebectomy hook of claim 24, wherein said illuminator comprises an electroluminescent device.

32. The phlebectomy hook of claim 31, wherein said electroluminescent device comprises an LED.
